(19) United States
(12) Patent Application Publication
(10) Pub. No.: US 2026/0027219 A1
(43) Pub. Date: Jan. 29, 2026

(54) PAL VARIANT, PHARMACEUTICAL COMPOSITION CONTAINING PAL VARIANT, AND METHOD FOR PREPARING PAL VARIANT

(71) Applicant: ZEKTAR BIOPHARMACEUTICALS (ZHEJIANG) CO., LTD., Hangzhou, Zhejiang (CN)

(72) Inventors: Zhefan YUAN, Hangzhou (CN); Huabao ZHENG, Hangzhou (CN); Ran WEI, Hangzhou (CN); Yangyang TANG, Hangzhou (CN); Chengdong JIN, Hangzhou (CN)

(73) Assignee: ZEKTAR BIOPHARMACEUTICALS (ZHEJIANG) CO., LTD., Hangzhou, Zhejiang (CN)

(21) Appl. No.: 18/996,453

(22) PCT Filed: Feb. 22, 2023
(86) PCT No.: PCT/CN2023/077574

§ 371 Date: Jan. 17, 2025

(30) Foreign Application Priority Data
Jul. 18, 2022 (CN) 202210839871.7

(51) Int. Cl. A61K 47/60; A61K 47/02; A61K 47/12; A61K 47/18; C12N 9/88

(52) U.S. Cl. CPC A61K 47/60; A61K 47/02; A61K 47/12; A61K 47/183; C12N 9/88; C12Y 403/01024

(57) ABSTRACT

The present disclosure provides a variant of phenylalanine ammonia lyase (PAL), the phenylalanine ammonia lyase (PAL) being derived from Anabaena variabilis and comprises the amino acid sequence shown in SEQ ID NO: 1, wherein the variant comprises amino acid substitution C503S/C565L, so that the variant comprises the amino acid sequence shown in SEQ ID NO: 2; or amino acid substitution C503S/C565P, so that the variant comprises the amino acid sequence shown in SEQ ID NO: 3; or amino acid substitution C503L/C565P, so that the variant comprises the amino acid sequence shown in SEQ ID NO: 4. The present disclosure also provides a conjugate and a pharmaceutical composition comprising the variant, a polynucleotide encoding the variant, a bacterium comprising the variant, and a method for preparing the variant.

### CROSS-REFERENCE TO RELATED APPLICATIONS

This application claims priority and benefits of Chinese patent application No. 202210839871.7 filed with the CNIPA on Jul. 18, 2022, the entire contents of which are incorporated herein by reference.

### TECHNICAL FIELD

The present disclosure provides a variant of phenylalanine ammonia lyase (PAL) derived from Anabaena variabilis, a conjugate and a pharmaceutical composition comprising the variant, a polynucleotide encoding the variant, a bacterium comprising the variant and a method for preparing the variant.

### BACKGROUND

Phenylalanine ammonia lyase (PAL) is a member of the aromatic amino acid lyase family (EC 4.3.1.23-1.25 and 4.3.1.3) together with histidine ammonia lyase (HAL) and tyrosine ammonia lyase (TAL). More specifically, enzymes with PAL activity (EC 4.3.1.23-1.25 and formerly classified as EC 4.3.1.5) catalyze the nonoxidative deamination of L-phenylalanine to trans-cinnamic acid. PAL is a non-mammalian enzyme that is widely distributed in plants, fungi and a limited number of bacteria. The PAL enzyme can be used as a therapeutic protein to treat the metabolic disorder phenylketonuria (PKU). PKU is an autosomal inherited disorder of metabolism in which the liver enzyme phenylalanine hydroxylase (PAH) or one or more enzymes involved in the synthesis or recycling of the cofactor tetrahydrobiopterin are deficient in function due to mutations in the corresponding genes. This lack of function results in high levels of phenylalanine in the bloodstream. Higher levels of phenylalanine are converted to phenylpyruvate (phenyl ketone) and other derivatives. If PKU is not treated promptly, high levels of phenylalanine and some of its breakdown products in the body can cause significant medical problems including intellectual disability, microcephaly, and seizures. Much research has focused on the use of PAL to treat PKU via enzyme replacement (Ambrus et al., Science 201:837-839 [1978]; Bourget et al., Appl Biochem. Biotechnol, 10:57-59 [1984]; and Sarkissian et al., Proc. Natl. Acad. Sci. USA 96:2339-2344 [1999]).

One approach for removing phenylalanine from serum is to use injectable recombinant PAL and PAL variants modified by pegylation (PEG-PAL). The PEGylation method has been shown to improve the half-life of the enzyme and reduce the antigenic response of the subject (see WO 2008/153776, WO 2011/097335 and U.S. Pat. No. 7,531,341). PAL variants that are effective in PEG-PAL compositions have been described as variants of wild-type Nostoc punctiforme (NpPAL); Anabaena variabilis (PAL); and Rhodosporidium toruloides (RtPAL). In particular, variants of wild-type PAL have been described in which the cysteine residues at positions 64, 318, 503 and 565 are replaced by serine (see U.S. Pat. Nos. 7,790,433; 7,560,263 and 7,537,923).

Although PAL has the potential for various therapeutic uses, factors such as reduced specific activity and proteolytic instability have limited the use of PAL. Similar to other therapeutic proteins, the use of PAL as an enzyme therapy is associated with several disadvantages, such as immunogenicity and proteolytic sensitivity (see Vellard, Curr. Opin. Biotechnol. 14:1-7 (2003)). Furthermore, a delicate balance between substrate affinity and enzyme activity is required to achieve and maintain plasma phenylalanine levels within a normal but somewhat narrow range in disorders characterized by hyperphenylalaninemia. So far, no concerted efforts aimed at improving these parameters have been made due to the lack of structural and biochemical knowledge about this protein.

Therefore, there remains a need for PAL molecules with optimal kinetic characteristics (including potent catalytic activity, longer biological half-life, greater biochemical stability and/or reduced immunogenicity) for therapeutic uses, such as the treatment of hyperphenylalaninemia (HPA, such as PKU) and other diseases (such as cancer).

### SUMMARY

In order to solve the above problems, the object of the present disclosure is to provide a phenylalanine ammonia lyase (PAL) variant according to the present disclosure or obtained by the method according to the present disclosure, which variant is a variant of Anabaena phenylalanine ammonia lyase (UniProtKB/Swiss Prot: Q3M5Z3.1) having the amino acid sequence shown in SEQ ID NO: 1, or the variant has the amino acid sequence shown in SEQ ID NO: 2, SEQ ID NO: 3, or SEQ ID NO: 4. The PAL variants according to the present disclosure may include one or more amino acid substitutions or combinations of amino acid substitutions, for example, a combination of one or more of the following: C503S/C565L, C503S/C565P, C503L/C565P mutations, and T102R, T102H, 1165L, 1165V, G166A, G166S, G218A, G218S, G218N, M222L, M222I, N437S, S438N, S438A, 1439V, 1439C, 1439A, 1439K, A440T, A440G, A440V, A440T, R441N, R442Q, T445V, T445S, A447S, E448K, N453G, Q457L, and G458K. In particular, the PAL variant enzyme according to the disclosure has phenylalanine ammonia lyase activity.

The PAL variant enzyme according to the disclosure or obtained by the method according to the disclosure can be used for the treatment of diseases or disorders characterized by elevated blood concentrations of phenylalanine. In particular, the PAL variant enzymes according to the disclosure can be used to treat phenylketonuria (PKU).

According to one embodiment of the present disclosure, the PAL variant enzyme according to the present disclosure has enhanced catalytic activity from phenylalanine to trans-cinnamic acid, enhanced thermal stability and low aggregation.

The definitions set forth below and throughout this application are used herein to describe the subject matter in accordance with the present disclosure.

### Definitions and Terminology

The disclosed subject matter may be further described using the following definitions and terminology. The definitions and terminology used herein are for the purpose of describing particular embodiments only and are not intended to be limiting. In the context of the present disclosure, “approximately”, “about”, “substantially”, and “significantly” will be understood by one of ordinary skill in the art and will depend to some extent on the context in which they are used. If the usage of a term is unclear to one of ordinary skill in the art in the context in which the term is used, “approximately” and “about” will mean plus or minus 10% of the particular term, while “substantially” and “significantly” will mean more than plus or minus 10% of the particular term.

In the context of the present disclosure, the terms “comprising” and “including” should be interpreted as open ended, allowing for the further inclusion of additional parts besides those listed. The terms “consisting of” and “composed of” should be interpreted as being closed and not allowing the inclusion of parts other than those listed in the claims. The term “consisting essentially of” should be interpreted as partially closed, i.e., only allowing the inclusion of additional ingredients that do not fundamentally change the properties of the claimed subject matter.

The modal verbs “may” or “can” refer to the preferred use or selection of one or more options or choices among the several embodiments or features described. Without disclosing an option or choice regarding a particular embodiment or feature contained therein, the modal verb “may” refers to an affirmative action regarding how to perform or use the embodiment or feature described. In the following context, the modal verb “may” has the same meaning and connotation as the auxiliary verb “J vX”.

In the context of the present disclosure, the following amino acid abbreviations are used:
Alanine: Ala(A) Arginine: Arg(R)
Asparagine: Asn(N) Aspartic acid: Asp(D)
Cysteine: Cys(C) Glutamine: Gln(Q)
Glutamic acid: Glu(E) Glycine: Gly(G)
Histidine: His(H) Isoleucine: IIe(I)
Leucine: Leu(L) Lysine: Lys(K)
Methionine: Met(M) Phenylalanine: Phe(F)
Proline: Pro(P) Serine: Ser(S)
Threonine: Thr(T) Tryptophan: Trp(W)
Tyrosine: Tyr(Y) Valine: Val(V)

In the context of the present disclosure, the terms “peptide”, “polypeptide” and “protein” refer to a molecule comprising a polymer of chains of amino acid residues linked by amide bonds. The term “amino acid residue” includes, but is not limited to, the amino acid residues contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (lie or l), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W) and tyrosine (Tyr or Y) residues. The term “amino acid residue” may also include non-standard or unnatural amino acids. The term “amino acid residue” may include α, β, γ and δ amino acids.

In the context of the present disclosure, “protein”, “polypeptide” and “peptide” are used interchangeably herein to refer to a polymer of at least two amino acids covalently linked by amide bonds, regardless of length or post-translational modifications (eg, glycosylation or phosphorylation).

An “analog” or “derivative” as used in the context of the present disclosure is a compound, such as a peptide, having a sequence similarity of greater than about 70% but less than 100% to a given compound, such as a peptide. These analogs or derivatives may contain non-naturally occurring amino acid residues as well as naturally occurring amino acid residues. These analogs or derivatives may also contain one or more D-amino acid residues and may contain non-peptide linkages between two or more amino acid residues.

In the context of the present disclosure, “polynucleotide” refers to a polymer composed of nucleotide units. Polynucleotides include naturally occurring nucleic acids, such as deoxyribonucleic acid (DNA) and ribonucleic acid (RNA), as well as nucleic acid analogs. Nucleic acid analogs include non-naturally occurring bases, nucleotides that participate in linkages with other nucleotides other than the naturally occurring phosphodiester bonds, or include bases linked by linkages other than phosphodiester bonds. Thus, nucleotide analogs include, for example but not limited to, phosphorothioates, phosphorodithioates, phosphotriesters, phosphoramidates, boranophosphates, methyl phosphonates, chiral methyl phosphonates, 2-O-methyl ribonucleotides, peptide-nucleic acids (PNAs), and the like. These polynucleotides can be synthesized, for example, using an automatic DNA synthesizer. The term “nucleic acid” generally refers to large polynucleotides. The term “oligonucleotide” generally refers to short polynucleotides, usually no longer than about 50 nucleotides. It should be understood that when a nucleotide sequence is expressed as a DNA sequence (ie, A, T, G, C), this also includes RNA sequences (ie, A, U, G, C), where “U” replaces “T”.

According to one embodiment of the present disclosure, there is provided a vector, such as an expression vector, containing a nucleic acid encoding one or more polypeptides and/or proteins according to the present disclosure.

In the context of the present disclosure, the term “encoding” refers to the inherent property of a specific sequence of nucleotides in a polynucleotide such as a gene, cDNA or mRNA as a template for the synthesis of other polymers and macromolecules in biological processes, which have a defined nucleotide sequence (i.e., rRNA, tRNA and mRNA) or a defined amino acid sequence and the biological properties resulting therefrom. Thus, if, in a cell or other biological system, transcription and translation of mRNA produced by a gene produces a protein, then the gene encodes that protein. Both the coding strand (whose nucleotide sequence is identical to the mRNA sequence and is typically provided in sequence listings) and the noncoding strand, which serves as the transcription template for a gene or cDNA, can be said to encode the protein or other product of the gene or cDNA. Unless otherwise specified, a “nucleotide sequence encoding an amino acid sequence” includes all nucleotide sequences that are degenerate forms of each other and that encode the same amino acid sequence. Nucleotide sequences encoding proteins and RNA can contain introns.

In the context of the present disclosure, the term “vector” refers to a nucleic acid molecule capable of transporting another nucleic acid to which it is linked, and “expression vector” refers to a vector comprising a recombinant polynucleotide comprising an expression control sequence operably linked to a nucleotide sequence to be expressed. The expression vector contains sufficient cis-acting elements for expression; other expression elements can be provided by the host cell or in vitro expression system. The expression vector includes all expression vectors known in the art, such as cosmids, plasmids (eg, naked or contained in liposomes), and viruses into which the recombinant polynucleotide has been integrated.

In the context of the present disclosure, the term “recombinant polynucleotide” refers to a polynucleotide having sequences that are not linked together in nature. The amplified or assembled recombinant polynucleotide can be contained in a suitable vector, and the vector can be used to transform a suitable host cell. Host cells that contain a recombinant polynucleotide are referred to as “recombinant host cells.” The gene is then expressed in a recombinant host cell to produce, for example, a “recombinant polypeptide.” A recombinant polynucleotide may also serve non-coding functions (eg, promoter, origin of replication, ribosome binding site, etc.).

In one embodiment according to the disclosure, the expression vector is a “plasmid”, which refers to a circular double-stranded DNA loop into which additional DNA segments can be ligated.

In the context of the present disclosure, “insertion” refers to the addition of one or more nucleotides to a native polynucleotide sequence. The engineered strains disclosed herein may include the insertion of one or more genes. In some embodiments, the engineered strains disclosed herein include the insertion of a sequence encoding an exogenous phenylalanine ammonia lyase (PAL). In some embodiments, the engineered strains disclosed herein include an insertion into an endogenous gene (ie, a genomic insertion) that results in a non-functional gene product.

In the context of the present disclosure, the term “substitution” refers to replacing a nucleotide of a native polynucleotide sequence with a nucleotide that is not native to the polynucleotide sequence. The engineered strains disclosed herein may include substitutions in one or more genes. In some embodiments, the substitution results in a non-functional gene product, for example, where the substitution introduces a premature stop codon (eg, TAA, TAG, or TGA) in the coding sequence of the gene product. In some embodiments according to the present disclosure, the engineered strain according to the present disclosure may comprise two or more substitutions, wherein the substitutions introduce multiple premature stop codons (eg, TAATAA, TAGTAG, or TGA).

In the context of the present disclosure, the term “primer” refers to a polynucleotide that is capable of specifically hybridizing to a designated polynucleotide template and providing a starting point for the synthesis of a complementary polynucleotide. Such synthesis occurs when the polynucleotide primer is placed under conditions that induce synthesis, ie, in the presence of nucleotides, a complementary polynucleotide template, and a polymerization agent such as a DNA polymerase. Primers are generally single-stranded, but can also be double-stranded. Primers are typically deoxyribonucleic acids, but many types of synthetic and naturally occurring primers are also useful in many applications. A primer is complementary to the template to which it is designed to hybridize to serve as a site for initiating synthesis, but need not reflect the exact sequence of the template. In this case, specific hybridization of the primer to the template depends on the stringency of the hybridization conditions. Primers can be labeled with, for example, a chromogenic, radioactive, or fluorescent moiety and used as a detectable moiety.

In the context of the present disclosure, the term “wild type” refers to the natural genetic form of an organism. The wild type is distinguished from a mutant form (an organism with a genetic mutation).

In the context of the present disclosure, “variants according to the present disclosure”, “PAL variants”, “variants of phenylalanine ammonia lyase”, “variant PAL”, “PAL variant enzyme” and “PAL enzyme variant” are used interchangeably.

In the context of the present disclosure, the term “effective amount” refers to a dosage sufficient to produce a desired result on a subject's health condition, pathology and disease or for diagnostic purposes. Desired results may include subjective or objective improvement in the recipient of the dose. A “therapeutically effective amount” is an amount of a drug effective to produce the desired beneficial effect on health. In any individual case, an appropriate “effective” amount may be determined by one skilled in the art using routine experimentation.

In the context of the present disclosure, the term “treatment” refers to prophylactic treatment or therapeutic treatment or diagnostic treatment.

In the context of the present disclosure, the term “pharmaceutical composition” refers to a composition suitable for use as a medicine in animal subjects including humans and mammals. The pharmaceutical composition comprises a pharmacologically effective amount of a PAL polypeptide and a pharmaceutically acceptable carrier. Pharmaceutical compositions include active ingredients and inert ingredients that constitute carriers, as well as any product produced directly or indirectly by the combination, complexation or aggregation of any two or more ingredients, or the dissociation of one or more ingredients, or other types of reactions or interactions of one or more ingredients. Therefore, the pharmaceutical compositions of the present disclosure include any compositions made by mixing the compound or conjugate of the present disclosure and a pharmaceutically acceptable carrier.

In the context of the present disclosure, the term “pharmaceutically acceptable carrier” refers to any of standard pharmaceutical carriers, buffers and excipients (e.g., phosphate buffered saline, 5% dextrose in water) and emulsions (e.g., water-in-oil or oil-in-water emulsions) and various types of wetting agents and/or adjuvants.

In a first aspect of the present disclosure, there is provided a PAL variant, a PAL variant enzyme, a PAL mutant or an analogue according to the present disclosure. According to one embodiment of the present disclosure, the PAL is derived from Anabaena variabilis and has the amino acid sequence shown in SEQ: ID NO: 1, and the PAL variant according to the present disclosure has an amino acid substitution selected from any one or a combination of two or more of the following: C503S/C565L, C503S/C565P, C503L/C565P, T102R, T102H, 1165L, 1165V, G166A, G166S, G218A, G218S, G 437S, S438N, S438A, 1439V, 1439C, 1439A, 1439K, A440T, A440G, A440V, A440T, R441N, R442Q, T445V, T445S, A447S, E448K, N453G, Q457L, and G458K. Preferably, the PAL variant enzyme according to the disclosure has phenylalanine ammonia lyase activity (EC 4.3.1.24). Preferably, the PAL variant enzyme according to the disclosure has enhanced phenylalanine ammonia lyase activity (EC 4.3.1.24) relative to the wild-type PAL enzyme. With the aid of the PAL variants according to the disclosure, an increase in the catalytic activity from phenylalanine to trans-cinnamic acid, an increase in the thermal stability and low aggregation are achieved.

According to one embodiment of the present disclosure, the PAL variant according to the present disclosure comprises the amino acid substitutions C503L/C565P/222L, or the amino acid substitutions C503L/C565P/G218A.

According to one embodiment of the present disclosure, in the reaction of converting phenylalanine into trans-cinnamic acid, the activity of the variant is higher than that of the phenylalanine ammonia lyase comprising the amino acid sequence shown in SEQ ID NO: 1.

According to one embodiment of the present disclosure, the variant exhibits the same stability or higher stability at a temperature in the range of 37° C. to 70° C. compared to the phenylalanine ammonia lyase comprising the amino acid sequence shown in SEQ ID NO: 1. In other words, at a temperature in the range of 37° C. to 70° C., the PAL variant according to the present disclosure has the same or higher, and preferably higher, stability than the wild-type PAL.

According to one embodiment of the present disclosure, the variant has low aggregation compared to the phenylalanine ammonia lyase comprising the amino acid sequence shown in SEQ ID NO:1.

According to one embodiment of the present disclosure, the PAL variant or derivative according to the present disclosure may have an amino acid sequence comprising conservative amino acid substitutions or non-conservative amino acid substitutions relative to the reference amino acid sequence SEQ: ID NO: 1. For example, a PAL variant or derivative peptide, polypeptide or protein according to the disclosure may include conservative amino acid substitutions and/or non-conservative amino acid substitutions relative to a reference peptide, polypeptide or protein. “Conservative amino acid substitutions” refer to substitutions that are expected to have the least effect on the properties of a reference peptide, polypeptide or protein. For example, each of the following six groups includes amino acids that are conservative substitutions for each other: (1) alanine (A), serine(S), threonine (T); (2) aspartic acid (D), glutamic acid (E); (3) asparagine (N), glutamic acid (Q); (4) arginine (R), lysine (K); (5) isoleucine (I), leucine (L), methionine (M), valine (V); and (6) phenylalanine (F), tyrosine (Y), tryptophan (W). “Non-conservative amino acid substitutions” are those substitutions that are predicted to most significantly perturb the properties of a reference peptide, polypeptide or protein. In other words, conservative amino acid substitutions substantially preserve the structure and function of the reference peptide, polypeptide or protein, whereas non-conservative amino acid substitutions do not preserve the structure and function of the reference peptide, polypeptide or protein.

According to one embodiment of the present disclosure, the PAL variant according to the present disclosure can be expressed in plants, fungi and bacteria.

According to a preferred embodiment of the present disclosure, the PAL variant according to the present disclosure can be expressed in Nostoc punctata, Anabaena variabilis, Rhodosporidium toruloides, Streptomyces maritima, Anacystis nidulans, Photorhabditis luminescens, Streptomyces verticillatus, and Escherichia coli.

According to one embodiment of the present disclosure, the PAL variant according to the present disclosure can be expressed in probiotics or engineered bacteria, which are capable of polynucleotide recombination and are not pathogenic bacteria and do not produce toxins. The PAL variant according to the present disclosure can be produced by introducing a recombinant polynucleotide encoding the PAL variant according to the present disclosure into the probiotic bacteria or engineered bacteria and expressing the polynucleotide. Examples of the probiotics or engineered bacteria include Escherichia coli and G⁺ bacteria, such as Bacillus subtilis.

In a second aspect of the disclosure, the disclosure provides a conjugate comprising a PAL variant according to the disclosure, wherein the conjugate comprises a PAL variant according to the disclosure and a suitable polymer, and in particular consists of a PAL variant according to the disclosure and a suitable water-soluble polymer. Here, the suitable water-soluble polymer includes polyethylene glycol, polyzwitterionic polymer, and polyoxazoline. According to a preferred embodiment of the present disclosure, the water-soluble polymer is polyethylene glycol.

In a third aspect of the present disclosure, the present disclosure provides a pharmaceutical composition comprising: (i) a PAL variant enzyme according to the present disclosure; and (ii) a suitable pharmaceutically acceptable carrier. According to one embodiment of the present disclosure, the pharmaceutical composition according to the present disclosure comprises: (i) a conjugate of a PAL variant enzyme according to the present disclosure; and (ii) a suitable pharmaceutically acceptable carrier. It is contemplated that the conjugate of the PAL variant enzyme comprises the PAL variant enzyme chemically coupled to a polyethylene glycol (PEG) polymer.

According to another embodiment of the present disclosure, the PAL variant in the pharmaceutical composition according to the present disclosure has at least 80%, 85%, 90%, 91%, 92%, 93%, 94%, 95%, 96%, 97%, 98% or 99% sequence identity with SEQ ID NO: 4.

According to one embodiment of the present disclosure, a pharmaceutical composition according to the present disclosure is formulated and administered to a subject in need thereof. Taking into account factors such as age, sex, weight and condition of a particular patient and the route of administration, the pharmaceutical composition according to the present disclosure can be formulated and/or administered according to dosages and techniques well known to those skilled in the medical field.

According to one embodiment of the present disclosure, the pharmaceutical composition according to the present disclosure comprises a pharmaceutically acceptable carrier, a diluent, an excipient and a surfactant. Additionally, the pharmaceutical composition may further include a preservative (eg, an antimicrobial or antibacterial agent such as benzalkonium chloride). According to a preferred embodiment of the present disclosure, the pharmaceutical composition may further comprise a buffer (eg, in order to maintain the pH of the composition between 6.5 and 7.5).

According to one embodiment of the present disclosure, the pharmaceutical composition according to the present disclosure can be used to treat phenylketonuria.

In the fourth aspect of the present disclosure, the present disclosure provides a polynucleotide for encoding the PAL variant according to the present disclosure, wherein the polynucleotide is DNA or RNA, in particular, the polynucleotide is cDNA or mRNA or tRNA. These DNAs and RNAs have specific nucleotide sequences, so that the PAL variant enzyme according to the present disclosure can be synthesized using them as templates.

According to one embodiment of the present disclosure, the polynucleotide according to the present disclosure is codon-optimized so that the polynucleotide can be used to express the PAL variant according to the present disclosure in E. coli. According to a preferred embodiment of the present disclosure, the polynucleotide according to the present disclosure is codon-optimized so that the polynucleotide can be used to express the PAL variant according to the present disclosure in animal cells, especially human cells.

According to a preferred embodiment of the present disclosure, the polynucleotide according to the present disclosure is codon-optimized so as to be able to express the PAL variant enzyme according to the present disclosure in E. coli. According to one embodiment of the present disclosure, the polynucleotide according to the present disclosure is codon-optimized so as to be able to express the PAL variant enzyme according to the present disclosure in human cells. The disclosed polynucleotides may be present in vectors known in the art (eg, plasmid vectors). According to one embodiment of the present disclosure, when the coding gene of the PAL enzyme variant is inserted into the vector pET28a, the base “GC” is introduced after the start codon of the restriction site Ncol, that is, a glycine is introduced after the first methionine to prevent frameshift during translation.

The PAL variants are, for example, variants of the Anabaena variabilis phenylalanine ammonia lyase PAL (SEQ ID NO: 1), for example, other PAL variants according to the present disclosure including PAL variants with amino acid sequences shown in SEQ ID NO: 2, SEQ ID NO: 3 and SEQ ID NO: 4.

In a fifth aspect of the present disclosure, an expression vector according to the present disclosure is provided, the expression vector comprises a polynucleotide according to the present disclosure, wherein the expression vector can be expressed in living cells.

According to one embodiment of the present disclosure, the expression vector according to the present disclosure includes all expression vectors known in the art, such as cosmids, plasmids (eg, naked or contained in liposomes) and viruses integrating recombinant polynucleotides.

According to a preferred embodiment of the present disclosure, the expression vector according to the present disclosure is a viral vector, such as adenovirus, adeno-associated virus and lentivirus.

According to another preferred embodiment of the present disclosure, the expression vector according to the present disclosure is a plasmid, which comprises a promoter, and the promoter is operably linked to the polynucleotide according to the present disclosure. According to another preferred embodiment of the present disclosure, the plasmid is transfected into the cell via liposome or cationic polymer.

In a sixth aspect, the present disclosure provides a bacterium according to the present disclosure, wherein the bacterium has integrated into its genome the polynucleotide according to the present disclosure and/or can express the PAL variant according to the present disclosure. Here, the present disclosure also provides a modified cell according to the present disclosure, which comprises a PAL variant according to the present disclosure and/or a polynucleotide according to the present disclosure and/or an expression vector according to the present disclosure, wherein optionally, the modified cell is an animal cell, especially a human cell.

In a host cell (e.g. a bacterium according to the disclosure or a modified cell according to the disclosure) comprising at least one polynucleotide encoding at least one PAL variant according to the disclosure, said polynucleotide is operably linked to one or more control sequences for expressing the PAL variant in the host cell. Suitable host cells for expressing the PAL variants according to the disclosure are, for example, bacteria according to the disclosure, such as Escherichia coli, Vibrio fluvialis, Streptomyces and Salmonella typhimurium cells; fungal cells, such as yeast cells (e.g., Saccharomyces cerevisiae or Pichia pastoris; insect cells, such as Drosophila S2 and Spodoptera Sf9 cells; animal cells, such as CHO, COS, BHK, 293 and Bowes melanoma cells; and plant cells. Exemplary host cells also include various Escherichia coli strains (e.g., W3110 (AfhuA) and BL21 (DE3)).

In its seventh aspect, the present disclosure provides a method for preparing a PAL variant according to the present disclosure, the method comprising culturing the bacterium according to the present disclosure in a culture medium or the modified cell according to the present disclosure to produce the PAL variant according to the present disclosure and isolating the PAL variant according to the present disclosure from the modified cell and/or the culture medium.

The PAL variant enzymes obtained by the process according to the disclosure can be used to treat diseases or disorders characterized by elevated blood concentrations of phenylalanine, such as phenylketonuria (PKU).

According to one embodiment of the present disclosure, the PAL variant enzyme obtained by the method according to the present disclosure compared to the PAL enzyme derived from Anabaena variabilis (SEQ ID NO: 1) has one or more amino acid substitutions selected from the group consisting of C503S/C565L, C503S/C565P, C503L/C565P mutations as well as T102R, T102H, 1165L, 1165V, G166A, G166S, G218A, G218S, G218N, M222L, M222I, N437S, S438N, S438A, 1439V, 1439C, 1439A, 1439K, A440T, A440G, A440V, A440T, R441N, R442Q, T445V, T445S, A447S, E448K, N453G, Q457L and G458K.

Specifically, according to one embodiment of the present disclosure, the method according to the present disclosure specifically comprises one or more of the following steps: (i) constructing a PAL original enzyme expression system including a plasmid, an expression cell or the like; (ii) extracting the pET28a-PAL plasmid from the PAL host cell; (iii) using the plasmid obtained in step (ii) as a template, constructing a mutant library using site-directed mutagenesis technology; (iv) cultivating PAL variants using a high-throughput screening method, and screening for superior mutations by detecting the enzyme activity of the variants.

The components in the context of the present disclosure may be crude and/or at least partially isolated and/or purified. In the context of the present disclosure, the term “isolated or purified” may refer to components that are removed from their natural environment and/or medium and are at least 60% free, preferably at least 75% free, and more preferably at least 90% free, even more preferably at least 95% free from other components and/or media with which they are naturally associated.

In the method for preparing the PAL variant according to the present disclosure, the PAL enzyme variants suitable for use in the method include, but are not limited to, variants of the PAL enzyme derived from Anabaena variabilis. Suitable transformed cells may include, but are not limited to, transformed E. coli, wherein the PAL gene sequence has preferably been codon-optimized to express the PAL enzyme variant according to the present disclosure in E. coli.

According to one embodiment of the present disclosure, the culture medium may be further supplemented and/or modified to promote the growth of transformed cells. According to one embodiment of the present disclosure, glucose is added to the culture medium, and the optional concentration is 0.1-0.3% (v/V).

According to one embodiment of the present disclosure, the transformed cells may be passaged one or more times. For example, transformed cells can be removed from the culture medium after the culture medium reaches an OD600 of at least about 1-2 (eg, when the transformed cells are still in the growth phase) and placed in fresh Minimum Essential Medium supplemented with phenylalanine. This optional subculturing step can be performed one or more times. Optionally, when the culture contains excess tCA, for example, when the culture medium contains at least 0.5, 1.0, 1.5, or 2.0 mM tCA, a subculturing step is performed to remove transformed cells from the culture.

In the eighth aspect of the present disclosure, the present disclosure provides a method for treating a disease or condition in a subject in need thereof, wherein the disease or condition is characterized by elevated levels of phenylalanine, wherein the method comprises administering to the subject the PAL variant according to the first aspect of the present disclosure; the conjugate according to the second aspect of the present disclosure; the pharmaceutical composition according to the third aspect of the present disclosure; the polynucleotide according to the fourth aspect of the present disclosure; the expression vector according to the fifth aspect of the present disclosure and/or the bacteria and modified cells according to the sixth aspect of the present disclosure.

According to one embodiment of the present disclosure, in the method for treating a disease or condition in a subject in need thereof according to the present disclosure, administration is performed by any suitable method known in the art, such as oral, transdermal, transmucosal, intrapulmonary (including aerosolization), intramuscular, subcutaneous or intravenous administration. According to a preferred embodiment of the present disclosure, the administration is subcutaneous or oral.

According to one embodiment of the disclosure, the dosage form is selected for oral or parenteral formulation in a manner known in the art. The pharmaceutical composition according to the present disclosure may be in the form of, for example, a solid, semisolid or liquid dosage form, such as a tablet, suppository, pill, capsule, powder, liquid, suspension, cream, ointment, lotion or the like, e.g., a unit dosage form suitable for single administration of a precise dose. The pharmaceutical composition according to the present disclosure may include a therapeutically effective amount of a PAL variant according to the present disclosure in combination with a pharmaceutically acceptable carrier; in addition, other agents, adjuvants, diluents, buffers, etc. may be optionally included.

According to one embodiment of the disclosure, a slow release or sustained release system may be employed so as to maintain a constant dosage level.

According to one embodiment of the present disclosure, the PAL variant according to the first aspect of the present disclosure or the conjugate according to the second aspect of the present disclosure is subcutaneously administered to the subject to treat diseases and disorders characterized by elevated levels of phenylalanine, in particular, the disease or disorder is phenylketonuria (PKU).

According to one embodiment of the present disclosure, the conjugate according to the second aspect of the present disclosure is subcutaneously administered to the subject, thereby increasing the half-life of the PAL variant enzyme when administered to a subject in need thereof. According to one embodiment of the disclosure, the conjugate comprises a PAL variant enzyme chemically coupled to a polyethylene glycol (PEG) polymer.

### BRIEF DESCRIPTION OF THE DRAWINGS

In order to make the above-mentioned objects, features and advantages of the present disclosure more easily understood, the specific embodiments of the present disclosure are described in detail below with reference to the accompanying drawings. In the following description, many specific details are set forth to provide a full understanding of the present disclosure. However, the present disclosure may also be implemented in other ways different from those described herein. Therefore, the present disclosure is not limited to the specific embodiments disclosed below.

FIG. 1 shows a schematic diagram of phenylalanine ammonia lyase (PAL) or a PAL variant catalyzing phenylalanine to produce trans-cinnamic acid;

FIG. 2A schematically shows the three-dimensional structure of a single subunit in a PAL tetramer;

FIG. 2B schematically shows the positions of some mutation sites in the three-dimensional structure;

FIGS. 3A and 3B schematically show the PAL high-throughput screening sites and amino acid substitutions with phenylalanine ammonia lyase activity according to the present disclosure;

FIG. 4 schematically shows the SEC-HPLC spectrum of PAL and its variants (PAL201: C503L/C565P; PAL202: G218A/C503L/C565P; PAL204: M222L/C503L/C565P) after combined purification (SEC chromatography column Ultrahydrogel 1000 (Waters), mobile phase: 0.3 ml/min PBS buffer solution);

FIG. 5 schematically shows an SDS-PAGE image of some PAL variants after affinity chromatography purification according to the present disclosure;

FIG. 6 schematically shows the changes in the enzyme activity of the PAL variant C503L/C565P according to the present disclosure at pH values between 6.5 and 9.0;

FIG. 7 schematically shows the changes in the thermal stability of the enzyme activities of the PAL variants according to the present disclosure and the wild-type PAL at a temperature of 37-70° C.;

FIG. 8 schematically shows the HPLC-SEC spectra of some PAL variants before and after modification with polyethylene glycol (PEG) according to the present disclosure.

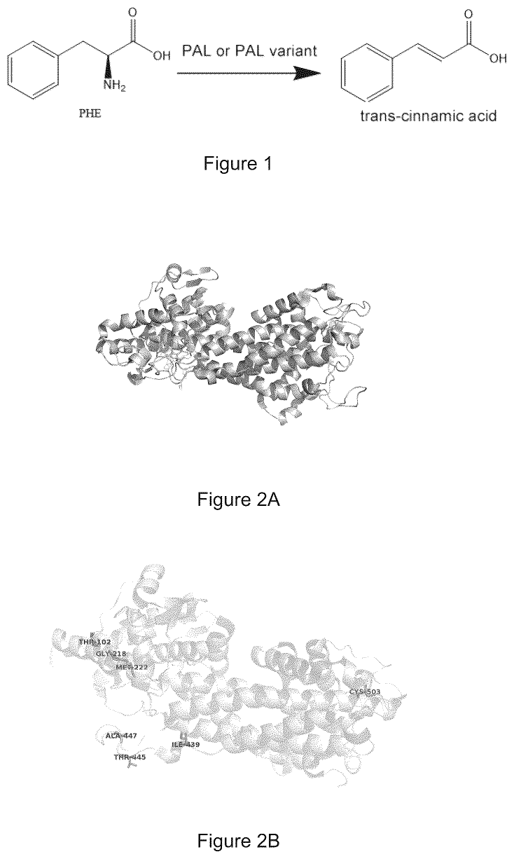

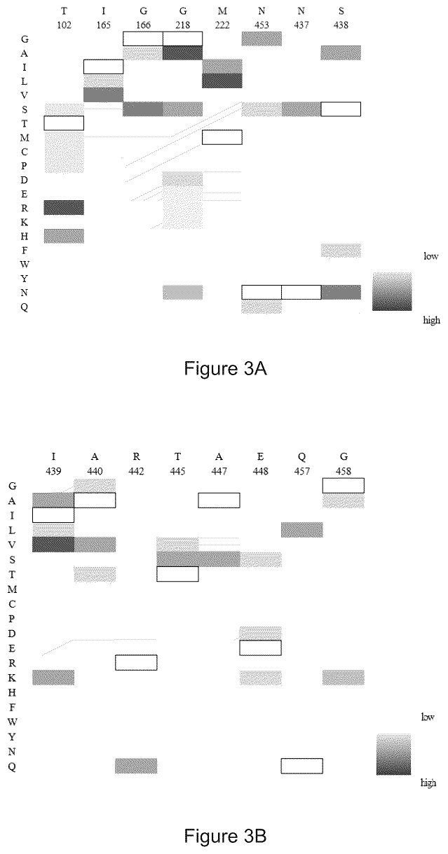

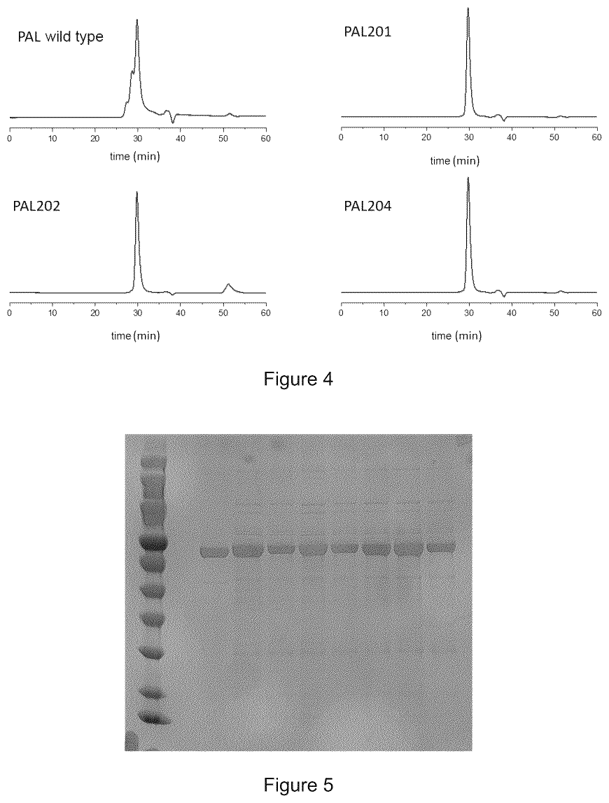

### DETAILED DESCRIPTION

The foregoing and other objects, elements and advantages will become apparent from the following more detailed description of specific embodiments, as illustrated and exemplified in the accompanying drawings. To the extent used, the terms “a”, “an” and singular forms of words as used in the claims and specification herein should be understood to include the plural form of the same word such that these terms indicate that one or more of something is provided. The terms “at least one” and “one or more” can be used interchangeably. The term “single” shall be used to indicate that one and only one of something is intended. Similarly, other specific integer values, such as “two”, are used when a specific number of something is required. The terms “preferably,” “preferred,” “preferably,” “optionally,” “may,” and similar terms are used to indicate that the recited items, conditions, or steps are optional (ie, not required) features of an embodiment. Unless otherwise stated, a range described as “between a and b” includes the values of “a” and “b.”

### Example 1 Construction of Genetically Engineered Bacteria E. coli BL21 (DE3)/pET28a-PAL

The phenylalanine ammonia lyase PAL encoding gene derived from Anabaena variabilis (amino acid sequence as shown in SEQ ID NO.1) was codon optimized, and the nucleotide sequence of the PAL gene after codon optimization was shown in SEQ ID NO: 2. The nucleotide sequence of the codon-optimized PAL gene (SEQ ID NO. 2) was artificially synthesized and inserted between Nco 1 and Xho 1 of pET28a to obtain the recombinant plasmid pET28a-PAL. The electrophoresis diagram is shown in FIG. 2.

Take out 5 μL of plasmid pET28a-PAL and add it to a 1.5 ml EP tube containing 50 μl E. coli BL21 (DE3) competent cells. Tap the tube wall to mix and place on ice for 30 min. After heat shock in a 42° C. water bath for 45 seconds, the cells were immediately placed on ice for 2 minutes. Add 1 mL of LB liquid culture medium to the EP tube and culture at 37° C. for 1 h. The culture solution was centrifuged at 4500 rpm for 4 min. 800 μL of the supernatant was taken out, the remaining supernatant was used to resuspend the bacteria, and 100 μL was taken out and spread on LB solid medium containing 50 μg/mL kanamycin. The bacteria were cultured at 37° C. for 12-14 hours to obtain the engineered bacteria E. coli BL21 (DE3)/pET28a-PAL expressing recombinant PAL.

LB liquid culture medium composition: yeast extract 5 g/L, tryptone 10 g/L, NaCl 10 g/L, solvent is distilled water, pH is about 7.0.

### Example 2 Construction of pET28a-PAL Saturated Mutation Plasmid and Expression Bacteria Containing the Mutation Plasmid

Using the plasmid pET28a-PAL prepared in Example 1 as a template, a pair of primers was designed, and the mutation site primer sequence was set to “NNN”. The whole plasmid was amplified by inverse PCR technology, and Dpnl was added to digest the plasmid. The plasmid was purified using a kit (TakaRa), and the plasmid was introduced into the cloning host bacteria DH5a by electroporation. After the plasmid was enriched by the cloning bacteria, the plasmid was extracted and then transformed into Escherichia coli E. coli BL21 (DE3). The primers for each site-saturation mutagenesis are shown in Table 1.

TABLE 1
Primers for site-saturation mutagenesis

| Primers | Sequence (5'-3') |
|---|---|
| T102X-F | AGCGAACTGCAGNNNAACCTGGTGTGGTTTCTGAAA |
| T102X-R | CCACACCAGGTTNNNCTGCAGTTCGCTTGCCTGTTC |
| I165X-F | GAATTTGGTAGCNNNGGTGCAAGCGGCGATCTG |
| I165X-R | GCCGCTTGCACCNNNGCTACCAAATTCATACAC |
| G166X-F | TTTGGTAGCATCNNNGCAAGCGGCGATCTGGTG |
| G166X-R | ATCGCCGCTTGCNNNGATGCTACCAAATTCATA |
| G218X-F | CTGCCTAAAGAANNNCTGGCAATGATGAACGGTACC |
| G218X-R | CATCATTGCCAGNNNTTCTTTAGGCAGCAGGGTCAG |
| M222X-F | GGTCTGGCAATGNNNAACGGTACCAGCGTGATGACC |
| M222X-R | GCTGGTACCGTTNNNCATTGCCAGACCTTCTTTAGG |
| G436X-F | CTGCTGACCTTTTATNNNAACAGCATTGCA |
| G436X-R | ATCTGCAATGCTGTTNNNATAAAAGGTCAG |
| N437X-F | CTGACCTTTTATGGTNNNAGCATTGCAGAT |
| N437X-R | ACGATCTGCAATGCTNNNACCATAAAAGGT |
| S438X-F | ACCTTTTATGGTAACNNNATTGCAGATCGT |
| S438X-R | AAAACGATCTGCAATNNNGTTACCATAAAA |
| 1439X-F | TTTTATGGTAACAGCNNNGCAGATCGTTTT |
| 1439X-R | AGGAAAACGATCTGCNNNGCTGTTACCATA |
| A440X-F | TATGGTAACAGCATTNNNGATCGTTTTCCT |
| A440X-R | GGTAGGAAAACGATCNNNAATGCTGTTACC |
| D441X-F | GGTAACAGCATTGCANNNCGTTTTCCTACC |
| D441X-R | ATGGGTAGGAAAACGNNNTGCAATGCTGTT |
| R442X-F | AACAGCATTGCAGATNNNTTTCCTACCCAT |
| R442X-R | TGCATGGGTAGGAAANNNATCTGCAATGCT |
| P444X-F | ATTGCAGATCGTTTTNNNACCCATGCAGAA |
| P444X-R | CTGTTCTGCATGGGTNNNAAAACGATCTGC |
| T445X-F | GCAGATCGTTTTCCTNNNCATGCAGAACAG |
| T445X-R | AAACTGTTCTGCATGNNNAGGAAAACGATC |
| H446X-F | GATCGTTTTCCTACCNNNGCAGAACAGTTT |
| H446X-R | GTTAAACTGTTCTGCNNNGGTAGGAAAACG |
| A447X-F | CGTTTTCCTACCCATNNNGAACAGTTTAAC |
| A447X-R | CTGGTTAAACTGTTCNNNATGGGTAGGAAA |
| E448X-F | CCTACCCATGCANNNCAGTTTAACCAGAACATCAAC |
| E448X-R | CTGGTTAAACTGNNNTGCATGGGTAGGAAAACGATC |
| Q449X-F | ACCCATGCAGAANNNTTTAACCAGAACATCAACAGC |
| Q449X-R | GTTCTGGTTAAANNNTTCTGCATGGGTAGGAAAACG |
| N451X-F | GCAGAACAGTTTNNNCAGAACATCAACAGCCAGGGT |
| N451X-R | GTTGATGTTCTGNNNAAACTGTTCTGCATGGGTAGG |
| N453X-F | CAGTTTAACCAGNNNATCAACAGCCAGGGTTATACC |
| N453X-R | CTGGCTGTTGATNNNCTGGTTAAACTGTTCTGCATG |
| 1454X-F | TTTAACCAGAACNNNAACAGCCAGGGTTATACCAGC |
| 1454X-R | ACCCTGGCTGTTNNNGTTCTGGTTAAACTGTTCTGC |
| N455X-F | AACCAGAACATCNNNAGCCAGGGTTATACCAGCGCA |
| N455X-R | ATAACCCTGGCTNNNGATGTTCTGGTTAAACTGTTC |
| S456X-F | CAGAACATCAACNNNCAGGGTTATACCAGCGCAACC |
| S456X-R | GGTATAACCCTGNNNGTTGATGTTCTGGTTAAACTG |
| Q457X-F | AACATCAACAGCNNNGGTTATACCAGCGCAACCCTG |
| Q457X-R | GCTGGTATAACCNNNGCTGTTGATGTTCTGGTTAAA |
| G458X-F | ATCAACAGCCAGNNNTATACCAGCGCAACCCTGGCA |
| G458X-R | TGCGCTGGTATANNNCTGGCTGTTGATGTTCTGGTT |
| C503X-F | ATGATGCACGTGCANNNCTGAGCCCTGCAACCG |
| C503X-R | GTTGCAGGGCTCAGNNNTGCACGTGCATCATAA |
| C565X-F | AGGATATTCTGCCTNNNCTGCATCTCGAGCACCA |
| C565X-R | TGCTCGAGATGCAGNNNAGGCAGAATATCCTGAA |

The inverse PCR amplification system is shown in Table 2.

TABLE 2
PCR amplification system

| Element | Dosage (ul) |
|---|---|
| 2 × PrimeSTAR ®(Premix) | 25 |
| Template plasmid | 1 |
| Primers | 2 each |
| dd H₂O | Up to 50 |

The PCR reaction process was as follows, and the PCR products were detected by 0.8% agarose gel electrophoresis.

TABLE 3
PCR amplification conditions

| | | |
|---|---|---|
| Temperature | Time | Cycle times |

| | | | | |
|---|---|---|---|---|
| 95° | C. | 5 | min | No cycles |
| 95° | C. | 15 | sec | 30 times |
| 55-65° | C. | 15 | sec | |
| 72° | C. | 2 | min | |

Take out 5 μL of the obtained saturated mutant plasmid and add it to 50 μL of E. coli Top10 competent medium, flick the tube wall to mix, and place on ice for 30 minutes. Heat shock in 42° C. water bath for 45 s and immediately place on ice for 2 min. Add 1 mL of LB liquid medium to the tube and culture at 37° C. for 1 h. The culture solution was centrifuged at 4500 rpm for 4 min, and 800 μL of the supernatant was taken out. The remaining supernatant was used to resuspend the bacteria, and 100 μL was taken out and applied on LB solid medium containing 100 μg/mL kanamycin. The cells were cultured at 37° C. for 12-16 hours to obtain clones containing the mutant plasmid. The plasmids were extracted after enrichment culture of the clones and transformed into E. coli BL21 (DE3) competent cells. The above transformation steps were repeated to obtain expression bacteria containing the mutant plasmid.

### Example 3: High-Throughput Screening of PAL Mutant Plasmid-Expressing Bacteria

### 1. Bacteria Culture

Pick up mutant plasmid expression bacteria (with or without His tag) from the plate, inoculate into a 96-deep-well plate with LB/Kana+ culture medium, culture at 37° C. and 800 rpm for 16 h, take out 100 μl and transfer to another 96-deep-well plate, each well has 900 μl TB culture medium, Kana and IPTG have been added, culture at 37° C. and 800 rpm for 16 h, then centrifuge at 3000 rpm at 4° C. for 30 min, remove the supernatant, and obtain wet bacterial cells.

### 2. Bacterial Cell Disruption

The wet bacterial cells in the 96-deep-well plate were frozen and thawed twice in a refrigerator at about −80° C., and then the bacterial cells were resuspended with Tris-HCl buffer (pH=8.5) containing lysozyme, shaken at 800 rpm for 30 min at 37° C., and centrifuged under the same conditions. The supernatant was the crude enzyme solution.

### 3. Enzyme Activity Detection and Enzyme Activity Calculation

Take 10 μl crude enzyme solution and add 190 μl Tris-HCl (100 mM, pH8.5) buffer containing 22.5 mM phenylalanine for measurement. Use the empty plasmid wells and PAL wells as the control group and the remaining wells as the experimental group. The measurement wavelength is 290 nm, and the parameters are set to measure absorbance. The measurement is 10 min in total, and the data is measured every 30s.

Definition of enzyme activity unit: Under the conditions of 37° C. and pH 8.5, 1 u mole of L-phenylalanine is converted into trans-cinnamic acid and NH₃ per minute, which is defined as 1 U.

The enzyme activity calculation formula is as follows:

$$Enzyme activity ( U / ml ) = Δ c \times V 1 / ( t \times V 2 )$$

Δc: product concentration change, unit: uM. By establishing a linear relationship between product concentration and absorbance, the detected product concentration change is obtained by substitution;
V1: total volume of the activity test system, in ml;
t: total duration of activity measurement, in minutes;
V2: volume of enzyme solution added, in ml

### 4. Comparison of Enzyme Activity Between PAL Variant Enzyme and Wild-Type PAL Enzyme

The comparison results are shown in Table 4, where “+” indicates that the enzyme activity of the PAL variant is higher than that of the wild-type PAL enzyme, “o” indicates that the enzyme activity is equal to that of the wild-type PAL enzyme, and “−” indicates that the enzyme activity is lower than that of the wild-type PAL enzyme.

TABLE 4
Comparison of enzyme activities of PAL variant
enzymes and wild-type PAL enzymes

| | Enzyme | | Enzyme |
|---|---|---|---|
| | activity | | activity |
| | compar- | | compar- |
| PAL variant enzyme | ison | PAL variant enzyme | ison |
| I165L/C503S/C565S | ∘ | I165L/C503L/C565P | + |
| I165V/C503S/C565S | + | I165V/C503L/C565P | + |
| G166A/C503S/C565S | ∘ | G166A/C503L/C565P | + |
| G166S/C503S/C565S | + | G166S/C503L/C565P | + |
| G218N/C503S/C565S | ∘ | G218N/C503L/C565P | + |
| M222I/C503S/C565S | ∘ | M222I/C503L/C565P | + |
| N437S/C503S/C565S | ∘ | N437S/C503L/C565P | + |
| S438N/C503S/C565S | ∘ | S438N/C503L/C565P | + |
| S438A/C503S/C565S | ∘ | S438A/C503L/C565P | + |
| I439V/C503S/C565S | + | I439V/C503L/C565P | + |
| I439C/C503S/C565S | − | I439C/C503L/C565P | − |
| I439A/C503S/C565S | + | I439A/C503L/C565P | + |
| I439K/C503S/C565S | − | I439K/C503L/C565P | − |
| A440T/C503S/C565S | − | A440T/C503L/C565P | ∘ |
| A440G/C503S/C565S | − | A440G/C503L/C565P | ∘ |
| A440V/C503S/C565S | − | A440V/C503L/C565P | + |
| A440T/C503S/C565S | − | A440T/C503L/C565P | ∘ |
| R441N/C503S/C565S | − | R441N/C503L/C565P | ∘ |
| R442Q/C503S/C565S | ∘ | R442Q/C503L/C565P | + |
| T445V/C503S/C565S | ∘ | T445V/C503L/C565P | + |
| T445S/C503S/C565S | ∘ | T445S/C503L/C565P | + |
| A447S/C503S/C565S | + | A447S/C503L/C565P | + |
| E448K/C503S/C565S | ∘ | E448K/C503L/C565P | ∘ |
| Q457L/C503S/C565S | ∘ | Q457L/C503L/C565P | ∘ |
| G458K/C503S/C565S | ∘ | G458K/C503L/C565P | ∘ |
| T102R/C503L/C565P | + | N453Q/C503L/C565P | + |
| T102H/C503L/C565P | ∘ | N453G/C503L/C565P | + |
| G218A/C503L/C565P | + | M222L/C503L/C565P | + |
| G218S/C503L/C565P | + | | |
| C503S/C565P | + | C503S/C565L | + |
| C503L/C565P | + | C503L/C565L | − |
| C503A/C565P | − | C503A/C565L | − |
| C503G/C565P | − | C503G/C565L | − |
| C503V/C565P | − | C503V/C565L | − |
| C503N/C565P | − | C503N/C565L | − |
| C503T/C565P | − | C503T/C565L | − |
| C503Q/C565P | − | C503Q/C565L | − |
| C503I/C565P | − | C503I/C565L | − |
| C503P/C565P | − | C503P/C565L | − |
| C503F/C565P | − | C503F/C565L | − |
| C503Y/C565P | − | C503Y/C565L | − |
| C503W/C565P | − | C503W/C565L | − |
| C503D/C565P | − | C503D/C565L | − |
| C503E/C565P | − | C503E/C565L | − |
| C503K/C565P | − | C503K/C565L | − |
| C503R/C565P | − | C503R/C565L | − |
| C503H/C565P | − | C503H/C565L | − |
| C503M/C565P | − | C503M/C565L | − |

### Example 4: PET28a-PAL and its Mutant Plasmid Expression Bacterial Cell Culture and Protein Induction

After pET28a-PAL and its mutant plasmids were transformed into E. coli BL21, wild-type PAL and PAL variant expression bacteria were obtained. Single colonies were picked from the plate and transferred to LB/Kana liquid medium. They were cultured overnight at 37° C. and 200 rpm for 12-16 h. The bacterial liquid was transferred to new LB/Kana liquid medium with an inoculation ratio of 2%. The culture was cultured at 37° C. and 200 rpm for 1-2 h. When OD600 was between 0.6 and 0.8, IPTG was added at a final concentration of 0.2 mM and induced at 30° C. and 200 rpm for 12-16 h.

After induction, the bacteria were collected by centrifugation at 6000 rpm for 10 min at 4° C. The wet cells were stored at −80° C.

### Example 5: Protein Specific Activity Determination

Protein concentration detection: The method for protein measurement was based on the BCA assay kit (Thermo), which is incorporated herein by reference; wherein the linear range is from 25 μg/ml to 2000 μg/ml. This embodiment uses a 96-well detection plate, and the volumes of the added enzyme solution and detection solution are 10 μl and 200 μl, or 25 μl and 200 μl, respectively.

Enzyme activity detection: The enzyme activity of wild-type PAL and PAL variants was detected using the enzyme activity detection method described in Example 3.

### Specific Activity Calculation:

Specific activity (U/mg)=enzyme activity (U/ml)/protein concentration (mg/ml)

### Example 6: Purification of Wild-Type PAL and PAL Variants

Cell lysis: The frozen cell pellet was thawed and resuspended in 20 mM Tris-HCl, 100 mM NaCl buffer at room temperature, pH 8.0, to form a cell slurry with a density of approximately 120 to 140 OD600. The cells were passed twice through a Niro NS30006 homogenizer at 700-800 Bar (where the temperature was controlled below 30° C.) to lyse the cells by homogenization. After homogenization, the pH was adjusted to about 8.0 by adding 1 N NaOH to the lysate. The cell lysate was gradually heated to 55° C., maintained at 55° C. for 30 to 120 min, and then cooled. Clarify cell lysate by centrifugation. Add ammonium sulfate to the supernatant of the lysate containing the target enzyme to a saturation of 30-70% and let stand overnight. Collect the precipitate by centrifugation. The enzyme precipitate was resuspended in Tris buffer solution at pH 8.5.

### Protein Purification:

### 1. PAL and its Variants Containing His Tag:

The Ni-NTA affinity chromatography column was balanced with 5 mM imidazole (50 mM Tris-HCl, pH 8.5, 0.5 M NaCl) for 2-5 column volumes; the cell lysate filtered through a 0.45 μm filter membrane or the above-mentioned Tris resuspension was loaded into the chromatography column; perform gradient elution with a buffer solution containing imidazole concentrations ranging from 5 mM to 200 mM (50 mM Tris-HCl, pH 8.5, 0.5 M NaCl), the elution peak was collected, and the molecular weight and purity of the fusion protein were detected by SDS-PAGE. The results of some PAL variants are shown in FIG.5, wherein FIG. 5 shows a total of 9 lanes, which are, from left to right, protein standard Marker (lane 1) and PAL variants containing a His tag (lanes 2 to 9), wherein lanes 2 to 9 represent, respectively: a PAL variant containing a C503L/C565P mutation, a PAL variant containing a mutation T102R/C503L/C565P, a PAL variant containing a G218A/C503L/C565P mutation, a PAL variant containing G218S/C503L/C565P mutations, PAL variant containing M222L/C503L/C565P mutations, a PAL variant containing S438N/C503L/C565P mutations, a PAL variant containing 1439V/C503L/C565P mutations; the standard sample bands shown therein are 200, 140, 95, 65, 52, 41, 33, 25, 17, and 10 from top to bottom. It can be seen that the target PAL variants appear between the relative molecular weights of 52-65 kDa;

2. PAL and its Variants without His Tag:

The Tris-resuspended enzyme solution was passed through a hydrophobic interaction (HIC) column and an anion exchange (AIEX) column in sequence to purify the PAL variant. It should be noted that the order of using the hydrophobic interaction (HIC) column and the anion exchange (AIEX) column can be interchanged, or only one chromatographic column can be used as a purification tool. It should be understood that other ALEX and HIC column resins may be used, and that the HIC column may be replaced by size exclusion chromatography. The precipitated resuspended enzyme solution was diluted 2 times with 50 mM Tris-HCl buffer solution with a pH value of 8.5, and loaded onto a TPGQ AIEX column equilibrated with 50 mM Tris-HCl buffer solution with a pH value of 8.5. The column was washed with a 50 mM Tris-HCl, 400 mM NaCl buffer solution at pH 8.5, followed by gradient elution of PAL using a Tris-HCl buffer (50 mM, pH 8.5) containing 0 to 1000 mM NaCl. Fractions with enzyme activity were collected, ultrafiltered and concentrated. Hydrophobic column chromatography: A HIC chromatography column (t-Butyl, Tosoh) was pre-equilibrated with a Tris-HCl buffer solution (50 mM, pH 8.5) containing 1 M (NH₄)₂SO₄. The PAL enzyme solution was diluted 2 times with a Tris-HCl buffer solution (50 mM, pH 8.5) containing 2 M (NH₄)₂SO₄, and loaded into the above-mentioned HIC chromatography column. It was gradient eluted with a Tris-HCl buffer solution (50 mM, pH 8.5) containing 1M to 0M (NH₄)₂SO₄, and the components with enzyme activity were collected and concentrated and desalted by ultrafiltration.

The specific activity data of the wild-type PAL enzyme and some PAL variant enzymes after purification are summarized in Table 5.

TABLE 5
Specific activity of wild-type PAL
enzyme and PAL variant enzyme

| PAL variant enzyme | Specific activity U/mg |
|---|---|
| Wild-type PAL enzyme | 1.1-1.6 |
| C503S/C565S | 1.8-2.2 |
| C503L/C565P | 1.9-3.0 |
| C503S/C565L | 1.8-2.3 |
| C503S/C565P | 1.9-2.4 |
| C503L/C565P/M222L | 2.1-4.3 |
| C503L/C565P/G218A | 2.6-3.2 |

### Example 7: Comparison of Aggregation of Wild-Type PAL Enzyme and PAL Variant Enzyme

The purified PAL was concentrated, and the concentrated protein solution (2.5 mg/ml) was incubated at 37° C. for 2 hours to accelerate the aggregation of the purified PAL protein in solution. Aggregation was examined by separating PAL protein by SEC-HPLC. To determine whether disulfide cross-linking was the cause of aggregation, 50 mM dithiothreitol (DTT) was added to the concentrated protein solution, followed by incubation at 37° C. for 2 hours.

The aggregation-prone wild-type PAL solution was further concentrated to approximately 10 mg/mL by ultrafiltration and incubated at 37° C. for 2 h. For aggregation-resistant PAL variants, the solution was further concentrated to approximately 30 mg/mL and incubated at 37° C. for 2 hours.

As shown in Table 6, the purified wild-type PAL enzyme aggregated after incubation at 37° C. for 2 hours. As expected, this aggregation was exacerbated when PAL protein was concentrated prior to incubation at 37° C. for 2 h. Exposure of concentrated proteins to DTT prevented aggregation, indicating that aggregation was caused by disulfide cross-linking. In contrast, the purified PAL variants (C503S/C565P) and PAL variants (C503S/C565L) did not form aggregates after incubation at 37° C. for 2 hours, indicating that the PAL variants have lower aggregation than the wild-type PAL enzyme.

TABLE 6
Effects of different treatments of
PAL variants on aggregate formation

| | | Aggre- |
|---|---|---|
| PAL protein | Treatment | gates |
| Wild-type PAL enzyme | 37° C./2 h | + |
| C503S/C565P | 37° C./2 h | − |
| C503S/C565L | 37° C./2 h | − |
| C503L/C565P | 37° C./2 h | − |
| C503L/C565P/G218A | 37° C./2 h | − |
| C503L/C565P/G218S | 37° C./2 h | − |
| C503L/C565P/M222L | 37° C./2 h | − |
| Wild-type PAL enzyme | Concentration + 37° C./2 h | ++ |
| C503S/C565P | Concentration + 37° C./2 h | − |
| C503S/C565L | Concentration + 37° C./2 h | − |
| C503L/C565P | Concentration + 37° C./2 h | − |
| C503L/C565P/G218A | Concentration + 37° C./2 h | − |
| C503L/C565P/G218S | Concentration + 37° C./2 h | − |
| C503L/C565P/M222L | Concentration + 37° C./2 h | − |
| Wild-type PAL enzyme | Concentration + DTT + 37° C./2 h | − |
| C503S/C565P | Concentration + DTT + 37° C./2 h | − |
| C503S/C565L | Concentration + DTT + 37° C./2 h | − |
| C503L/C565P | Concentration + DTT + 37° C./2 h | − |
| C503L/C565P/G218A | Concentration + DTT + 37° C./2 h | − |
| C503L/C565P/G218S | Concentration + DTT + 37° C./2 h | − |
| C503L/C565P/M222L | Concentration + DTT + 37° C./2 h | − |

“−” indicates no aggregation, “+” indicates aggregation, and “++” indicates severe aggregation.

### Example 8: PH Adaptation Curves of Wild-Type PAL Enzyme and PAL Variant Enzyme

The pH optima of the wild-type PAL enzyme and selected PAL variants were determined by the specific activity assay described in Example 5. Take 10 μl pure enzyme solution (total protein 0.2 μg)+190 μl 100 mM Tris-HCl (pH8.5) buffer dissolved with 22.5 mM Phe for determination, and perform enzyme activity detection in 200 μL system at 37° C.

The pH values of 100 mM Tris-HCl (pH 8.5) buffer dissolved with 22.5 mM Phe were set to 6.5, 7.0, 7.5, 8.0, 8.5, and 9.0, respectively.

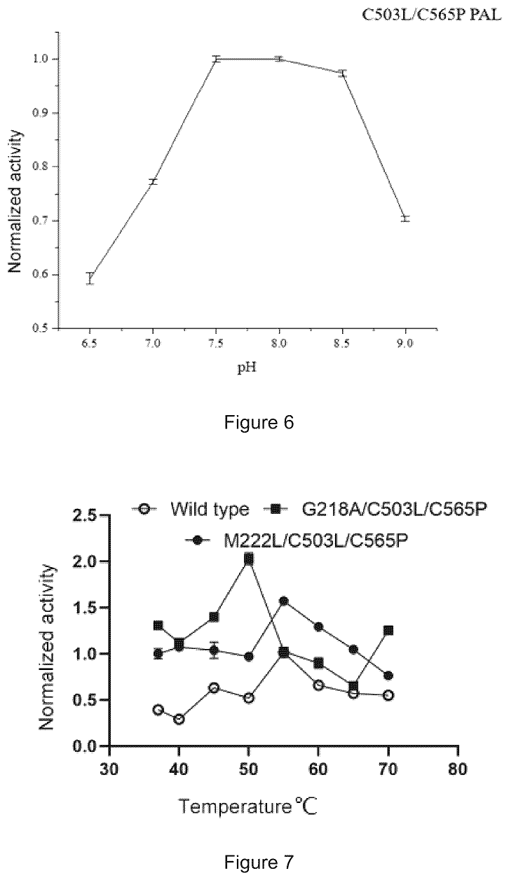

The measurement results are summarized in FIG. 6, where all results were normalized to the enzyme activity of the PAL variant with C503L/C565P mutations at pH 7.5. As can be seen from FIG. 6, the PAL variant having the C503L/C565P mutations has the best enzyme activity in the pH range of 7.5 to 8.5.

### Example 9: Temperature Stability of Wild-Type PAL Enzyme and PAL Variant Enzyme

The effect of temperature on the stability of wild-type PAL enzyme and selected PAL variant enzymes was determined by incubating the proteins in 50 mM Tris-HCl, PH 7.5 for 2 hours at temperatures ranging from 37° C. to 70° C. and then measuring enzyme activity. Each enzyme reaction used 1 μg of PAL protein and 22.5 mM phenylalanine as substrates in a total reaction volume of 200 UL at 37° C.

The measurement results are summarized in FIG. 7. All results were normalized to the enzyme activity of the PAL variant with M222L/C503L/C565P mutations at 37° C.

### Example 10: PEG-Modified PAL Variants

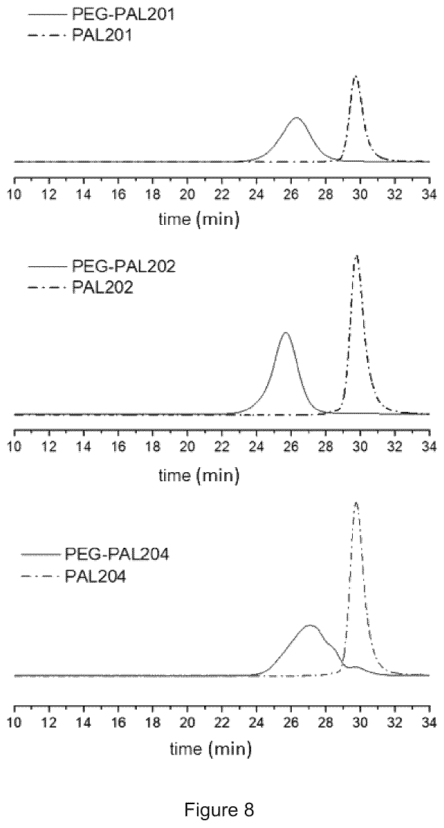

The solution containing PAL protein was dialyzed against PBS buffer, pH=8.0, 10 mM, to a fixed final protein concentration of 2.5 mg/ml. PEG (PEG-NHS) with a molecular weight of 20 kDa was added to the above PAL solution to fix the final PEG concentration at 75 mg/ml. The reaction solution of PEG and PAL is reacted at room temperature for 2-4 hours, or at 4° C. overnight. When the reaction was terminated, 10 μl of the above reaction solution was immediately taken for dilution, and the enzyme activity was determined according to the method in Example 5, and the change in enzyme activity before and after the reaction of PAL and PEG was calculated (see Table 7). The reaction solution containing the PEG-PAL conjugate was placed in a dialysis bag (molecular weight cut-off: 300 kD) and dialyzed extensively against a PBS 7.4 buffer solution. The final product was detected by HPLC-SEC. As shown in FIG. 8, after PEG modification, the three PAL variants all showed an obvious left shift in the HPLC-SEC spectrum, indicating that the PAL variants were successfully conjugated with PEG and the overall molecular size was significantly increased.

TABLE 7
Changes in enzyme activity of PAL
variants after PEG modification

| | Enzyme activity |
|---|---|
| Sample name | retention (%) |
| PEG-PAL201 | 91.0 |
| PEG-PAL202 | 106.6 |
| PEG-PAL204 | 92.6 |

It is worth noting that in embodiments according to the present disclosure, the steps of the method can be performed in any suitable order unless otherwise indicated herein or clearly contradicted by context. Unless otherwise indicated herein or clearly contradicted by context, these steps may be repeated any number of times to achieve the intended goal.

The use of any and all examples, or exemplary language (eg, “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred aspects of this disclosure are described herein, including the best mode known to the inventors for carrying out the disclosure. Variations of those preferences may become apparent to those of ordinary skill in the art upon reading the foregoing description. The inventors expect skilled artisans to employ such variations as appropriate, and the inventors intend for the disclosure to be practiced otherwise than as specifically described herein.

Although various improvements have been described herein with reference to specific embodiments of the disclosure, it should be understood that such description is by way of illustration only and should not be construed as limiting the scope of any claimed disclosure. Accordingly, the scope and content of any claimed disclosure will be determined solely by the terms of the appended claims in their current form or as amended during prosecution or as implemented in any continuation application. Furthermore, it should be understood that, unless otherwise stated, features of any specific embodiment discussed herein may be combined with one or more features of any one or more embodiments otherwise discussed or considered herein. Those skilled in the art will recognize that certain modifications and changes may be made to the described embodiments without departing from the spirit and scope of the present application as described in the appended claims, and that these modifications and changes fall within the scope of protection of the present disclosure.

Sequence table

SEQ ID NO 1:
MKTLSQAQSKTSSQQFSFTGNSSANVIIGNQKLTINDVARVARNG
TLVSLTNNTDILQGIQASCDYINNAVESGEPIYGVTSGFGGMANV
AISREQASELQTNLVWFLKTGAGNKLPLADVRAAMLLRANSHMRG
ASGIRLELIKRMEIFLNAGVTPYVYEFGSIGASGDLVPLSYITGS
LIGLDPSFKVDFNGKEMDAPTALRQLNLSPLTLLPKEGLAMMNGT
SVMTGIAANCVYDTQILTAIAMGVHALDIQALNGTNQSFHPFIHN
SKPHPGQLWAADQMISLLANSQLVRDELDGKHDYRDHELIQDRYS
LRCLPQYLGPIVDGISQIAKQIEIEINSVTDNPLIDVDNQASYHG
GNFLGQYVGMGMDHLRYYIGLLAKHLDVQIALLASPEFSNGLPPS
LLGNRERKVNMGLKGLQICGNSIMPLLTFYGNSIADRFPTHAEQF
NQNINSQGYTSATLARRSVDIFQNYVAIALMFGVQAVDLRTYKKT
GHYDARACLSPATERLYSAVRHVVGQKPTSDRPYIWNDNEQGLDE
HIARISADIAAGGVIVQAVQDILPCLH
SEQ ID NO 2:
MKTLSQAQSKTSSQQFSFTGNSSANVIIGNQKLTINDVARVARNG
TLVSLTNNTDILQGIQASCDYINNAVESGEPIYGVTSGFGGMANV
AISREQASELQTNLVWFLKTGAGNKLPLADVRAAMLLRANSHMRG
ASGIRLELIKRMEIFLNAGVTPYVYEFGSIGASGDLVPLSYITGS
LIGLDPSFKVDFNGKEMDAPTALRQLNLSPLTLLPKEGLAMMNGT
SVMTGIAANCVYDTQILTAIAMGVHALDIQALNGTNQSFHPFIHN
SKPHPGQLWAADQMISLLANSQLVRDELDGKHDYRDHELIQDRYS
LRCLPQYLGPIVDGISQIAKQIEIEINSVTDNPLIDVDNQASYHG
GNFLGQYVGMGMDHLRYYIGLLAKHLDVQIALLASPEFSNGLPPS
LLGNRERKVNMGLKGLQICGNSIMPLLTFYGNSIADRFPTHAEQF
NQNINSQGYTSATLARRSVDIFQNYVAIALMFGVQAVDLRTYKKT
GHYDARASLSPATERLYSAVRHVVGQKPTSDRPYIWNDNEQGLDE
HIARISADIAAGGVIVQAVQDILPLLH
SEQ ID NO 3:
MKTLSQAQSKTSSQQFSFTGNSSANVIIGNQKLTINDVARVARNG
TLVSLTNNTDILQGIQASCDYINNAVESGEPIYGVTSGFGGMANV
AISREQASELQTNLVWFLKTGAGNKLPLADVRAAMLLRANSHMRG
ASGIRLELIKRMEIFLNAGVTPYVYEFGSIGASGDLVPLSYITGS
LIGLDPSFKVDFNGKEMDAPTALRQLNLSPLTLLPKEGLAMMNGT
SVMTGIAANCVYDTQILTAIAMGVHALDIQALNGTNQSFHPFIHN
SKPHPGQLWAADQMISLLANSQLVRDELDGKHDYRDHELIQDRYS
LRCLPQYLGPIVDGISQIAKQIEIEINSVTDNPLIDVDNQASYHG
GNFLGQYVGMGMDHLRYYIGLLAKHLDVQIALLASPEFSNGLPPS
LLGNRERKVNMGLKGLQICGNSIMPLLTFYGNSIADRFPTHAEQF
NQNINSQGYTSATLARRSVDIFQNYVAIALMFGVQAVDLRTYKKT
GHYDARASLSPATERLYSAVRHVVGQKPTSDRPYIWNDNEQGLDE
HIARISADIAAGGVIVQAVQDILPPLH
SEQ ID NO 4:
MKTLSQAQSKTSSQQFSFTGNSSANVIIGNQKLTINDVARVARNG
TLVSLTNNTDILQGIQASCDYINNAVESGEPIYGVTSGFGGMANV
AISREQASELQTNLVWFLKTGAGNKLPLADVRAAMLLRANSHMRG
ASGIRLELIKRMEIFLNAGVTPYVYEFGSIGASGDLVPLSYITGS
LIGLDPSFKVDFNGKEMDAPTALRQLNLSPLTLLPKEGLAMMNGT
SVMTGIAANCVYDTQILTAIAMGVHALDIQALNGTNQSFHPFIHN
SKPHPGQLWAADQMISLLANSQLVRDELDGKHDYRDHELIQDRYS
LRCLPQYLGPIVDGISQIAKQIEIEINSVTDNPLIDVDNQASYHG
GNFLGQYVGMGMDHLRYYIGLLAKHLDVQIALLASPEFSNGLPPS
LLGNRERKVNMGLKGLQICGNSIMPLLTFYGNSIADRFPTHAEQF
NQNINSQGYTSATLARRSVDIFQNYVAIALMFGVQAVDLRTYKKT
GHYDARALLSPATERLYSAVRHVVGQKPTSDRPYIWNDNEQGLDE
HIARISADIAAGGVIVQAVQDILPPLH

1-28. (canceled)

29. A variant of phenylalanine ammonia lyase (PAL), the phenylalanine ammonia lyase (PAL) being derived from Anabaena variabilis and comprising the amino acid sequence shown in SEQ ID NO: 1, wherein the variant comprises:
amino acid substitution C503S/C565L, so that the variant comprises the amino acid sequence shown in SEQ ID NO: 2; or
amino acid substitution C503S/C565P, so that the variant comprises the amino acid sequence shown in SEQ ID NO: 3; or
amino acid substitution C503L/C565P, so that the variant comprises the amino acid sequence shown in SEQ ID NO: 4.

30. The variant of phenylalanine ammonia lyase according to claim 29, wherein the variant further comprises one or more mutations at the following amino acid substitution positions: T102R, T102H, 1165L, 1165V, G166A, G166S, G218A, G218S, G218N, M222L, M222I, N437S, S438N, S438A, 1439V, 1439C, 1439A, I439K, A440T, A440G, A440V, A440T, R441N, R442Q, T445V, T445S, A447S, E448K, N453G, Q457L and G458K.

31. The variant of phenylalanine ammonia lyase according to claim 29, wherein the variant comprises:
amino acid substitutions C503L/C565P/222L, or amino acid substitutions C503L/C565P/G218A.

32. The variant of phenylalanine ammonia lyase according to claim 29, wherein, in the reaction of converting phenylalanine into trans-cinnamic acid, the activity of the variant is higher than that of the phenylalanine ammonia lyase comprising the amino acid sequence shown in SEQ ID NO: 1.

33. The variant of phenylalanine ammonia lyase according to claim 30, wherein, in the reaction of converting phenylalanine into trans-cinnamic acid, the activity of the variant is higher than that of the phenylalanine ammonia lyase comprising the amino acid sequence shown in SEQ ID NO: 1.

34. The variant of phenylalanine ammonia lyase according to claim 29, wherein the variant exhibits the same stability or higher stability at a temperature ranging from 37° C. to 70° C. compared with the phenylalanine ammonia lyase comprising the amino acid sequence shown in SEQ ID NO:1.

35. The variant of phenylalanine ammonia lyase according to claim 30, wherein the variant exhibits the same stability or higher stability at a temperature ranging from 37° C. to 70° C. compared with the phenylalanine ammonia lyase comprising the amino acid sequence shown in SEQ ID NO:1.

36. The variant of phenylalanine ammonia lyase according to claim 29, wherein the variant has low aggregation compared with the phenylalanine ammonia lyase comprising the amino acid sequence shown in SEQ ID NO: 1.

37. The variant of phenylalanine ammonia lyase according to claim 30, wherein the variant has low aggregation compared with the phenylalanine ammonia lyase comprising the amino acid sequence shown in SEQ ID NO: 1.

38. The variant of the phenylalanine ammonia lyase according to claim 29, wherein the variant can be expressed in plants, fungi and bacteria.

39. The variant of phenylalanine ammonia lyase according to claim 29, wherein the variant can be expressed in Nostoc punctata, Anabaena variabilis, Rhodosporidium toruloides, Streptomyces maritima, Anacystis nidulans, Photorhabditis luminescens, Streptomyces verticillatus, or Escherichia coli.

40. A conjugate, comprising a variant of phenylalanine ammonia lyase (PAL), the phenylalanine ammonia lyase (PAL) being derived from Anabaena variabilis and comprising the amino acid sequence shown in SEQ ID NO: 1, wherein the variant comprises:
amino acid substitution C503S/C565L, so that the variant comprises the amino acid sequence shown in SEQ ID NO: 2; or
amino acid substitution C503S/C565P, so that the variant comprises the amino acid sequence shown in SEQ ID NO: 3; or
amino acid substitution C503L/C565P, so that the variant comprises the amino acid sequence shown in SEQ ID NO: 4; and a polymer, wherein the polymer is a water-soluble polymer.

41. The conjugate according to claim 40, wherein the water-soluble polymer is any one of polyethylene glycol, polyzwitterionic polymer and polyoxazoline, or a mixture of two or more thereof.

42. The conjugate according to claim 40, wherein the variant further comprises one or more mutations at the following amino acid substitution positions: T102R, T102H, 1165L, 1165V, G166A, G166S, G218A, G218S, G218N, M222L, M222I, N437S, S438N, S438A, 1439V, 1439C, 1439A, 1439K, A440T, A440G, A440V, A440T, R441N, R442Q, T445V, T445S, A447S, E448K, N453G, Q457L and G458K.

43. A pharmaceutical composition, comprising:
(i) a variant of phenylalanine ammonia lyase (PAL), the phenylalanine ammonia lyase (PAL) being derived from Anabaena variabilis and comprising the amino acid sequence shown in SEQ ID NO: 1, wherein the variant comprises:
amino acid substitution C503S/C565L, so that the variant comprises the amino acid sequence shown in SEQ ID NO: 2; or
amino acid substitution C503S/C565P, so that the variant comprises the amino acid sequence shown in SEQ ID NO: 3; or
amino acid substitution C503L/C565P, so that the variant comprises the amino acid sequence shown in SEQ ID NO: 4;
or a conjugate, comprising;
a variant of phenylalanine ammonia lyase (PAL), the phenylalanine ammonia lyase (PAL) being derived from Anabaena variabilis and comprising the amino acid sequence shown in SEQ ID NO: 1, wherein the variant comprises:
amino acid substitution C503S/C565L, so that the variant comprises the amino acid sequence shown in SEQ ID NO: 2; or
amino acid substitution C503S/C565P, so that the variant comprises the amino acid sequence shown in SEQ ID NO: 3; or
amino acid substitution C503L/C565P, so that the variant comprises the amino acid sequence shown in SEQ ID NO: 4;
and
a polymer, wherein the polymer is a water-soluble polymer; and
(ii) a suitable pharmaceutically acceptable carrier, which is any one or a mixture of two or more selected from the group consisting of Tris-HCl, NaCl, L-phenylalanine (L-Phe), glycine (Gly) and trans-cinnamic acid.

44. The pharmaceutical composition according to claim 43, wherein the pharmaceutical composition can be used to treat phenylketonuria.

45. The pharmaceutical composition according to claim 43, wherein the variant further comprises one or more mutations at the following amino acid substitution positions: T102R, T102H, 1165L, 1165V, G166A, G166S, G218A, G218S, G218N, M222L, M222I, N437S, S438N, S438A, 1439V, 1439C, I439A, I439K, A440T, A440G, A440V, A440T, R441N, R442Q, T445V, T445S, A447S, E448K, N453G, Q457L and G458K.

46. A modified cell comprising:
a variant of phenylalanine ammonia lyase (PAL), the phenylalanine ammonia lyase (PAL) being derived from Anabaena variabilis and comprising the amino acid sequence shown in SEQ ID NO: 1, wherein the variant comprises:
amino acid substitution C503S/C565L, so that the variant comprises the amino acid sequence shown in SEQ ID NO: 2; or
amino acid substitution C503S/C565P, so that the variant comprises the amino acid sequence shown in SEQ ID NO: 3; or
amino acid substitution C503L/C565P, so that the variant comprises the amino acid sequence shown in SEQ ID NO: 4;
a polynucleotide encoding a variant of the phenylalanine ammonia lyase, wherein the polynucleotide is DNA or RNA;
an expression vector comprises the polynucleotide, wherein the expression vector can be expressed in living cells; and
optionally, the modified cell is a modified animal cell.

47. The modified cell according to claim 46, wherein the modified cell is a human cell.

48. The modified cell according to claim 46, wherein the variant further comprises one or more mutations at the following amino acid substitution positions: T102R, T102H, 1165L, 1165V, G166A, G166S, G218A, G218S, G218N, M222L, M222I, N437S, S438N, S438A, I439V, 1439C, 1439A, I439K, A440T, A440G, A440V, A440T, R441N, R442Q, T445V, T445S, A447S, E448K, N453G, Q457L and G458K.


SEQUENCE LISTING (4 sequences)

SEQ ID NO: 1 - protein, 567 aa, Anabaena variabilis
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 2 - protein, 567 aa, Anabaena variabilis
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARASLSPATERLYSAVRHVVGQKPTSDRPYIWNDNEQGLDEHIARISADIAAGGVIVQAVQDILPLLH

SEQ ID NO: 3 - protein, 567 aa, Anabaena variabilis
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARASLSPATERLYSAVRHVVGQKPTSDRPYIWNDNEQGLDEHIARISADIAAGGVIVQAVQDILPPLH

SEQ ID NO: 4 - protein, 567 aa, Anabaena variabilis
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARALLSPATERLYSAVRHVVGQKPTSDRPYIWNDNEQGLDEHIARISADIAAGGVIVQAVQDILPPLH